(19) United States
(12) Patent Application Publication
(10) Pub. No.: US 2022/0155230 A1
(43) Pub. Date: May 19, 2022

(54) LIGHT SOURCE ACCOMMODATION FOR DIFFERENT SAMPLE MATRICES

(71) Applicant: PROCISEDX INC., San Diego, CA (US)

(72) Inventors: MICHAEL HALE, San Diego, CA (US); STEFAN WESTIN, San Diego, CA (US); RAJ SRIKRISHNAN, San Diego, CA (US); RUKMINI REDDY, San Diego, CA (US)

(73) Assignee: PROCISEDX INC., San Diego, CA (US)

(21) Appl. No.: 17/545,873

(22) Filed: Dec. 8, 2021

Related U.S. Application Data
(63) Continuation of application No. PCT/US2020/039819, filed on Jun. 26, 2020.
(60) Provisional application No. 62/868,661, filed on Jun. 28, 2019.

(51) Int. Cl. G01N 21/64; G01N 21/76; G01N 33/72

(52) U.S. Cl. CPC G01N 21/6452; G01N 33/721; G01N 21/76; G01N 21/6486

(57) ABSTRACT

Provided herein are methods for accommodating for sample matrix effects in light measurement assays. A selected amount of a light emitting material is added to a sample, and the light output from the sample is measured. By using an algorithm based on the known light emitting material amount and the measured light output, a correction factor for the assay of the sample is determined. The correction factor can be used to adjust subsequent light output measurements of other samples recorded using the assay.

### CROSS-REFERENCES TO RELATED APPLICATIONS

This application is a continuation of PCT/US2020/039819, filed Jun. 26, 2020, which application claims priority to U.S. Provisional Patent Application No. 62/868,661, filed Jun. 28, 2019, the teachings of which are hereby incorporated by reference in their entireties for all purposes.

### BACKGROUND

For many analytical techniques used to measure the presence, quantity, or identity of substances of interest in a sample, it is well understood that background components in the sample, i.e., the makeup of the sample matrix, can have a significant effect on both the way the analysis is to be performed and the quality of the results obtained. For example, sample matrix properties such as ionic strength and pH can affect the conformation or protonation state of a studied compound. Chelating agents, proteases, and inhibitors can interfere with required activities of enzymes associated with particular analytical workflows. Other materials in the sample matrix could exhibit properties similar to that of the analytes, confounding measurements and making it difficult to ascertain the underlying origin of observations. In each of these cases, false positive or false negative results can prevent the assay from achieving the accuracy or precision required for a given application.

For analytical protocols relying on the observation and quantification of light arriving at a detector from a sample, sample matrix effects can be similarly important to consider. In some cases, the turbidity of a sample can reduce the transmission of light through the sample before it reaches the detector. Light output from a sample can also be decreased if one or more elements of the sample matrix have a quenching effect on light of the observed wavelengths. Alternatively, some samples may have components that amplify or redirect light such that assay results can be exaggerated relative to readings taken in the absence of these components. Variations in sample matrix volume can additionally impact analytical findings even in cases in which the sample matrix composition is unchanged.

Most conventional approaches to addressing sample matrix effects in light measuring assays exclusively involve the use of external light sources. For example, a transillumination or epi-illumination light source can be used to project light onto or through a sample having an unknown or undetermined matrix, and the transiting, reflected, or emitted light is measured. The procedure is repeated for a reference sample having a known matrix, and the sample and reference data are compared. Because these techniques only use light sources that are outside of the sample and are part of the assay equipment, the derived sample compensations are closely tied to individual instruments. Additionally, these procedures can have a more limited applicability to sample types for which the measured light associated with the analytes of interest originates from within the sample itself.

The need therefore exists for methods that accommodate and compensate for sample matrix differences among analytical samples. Compensation for differences and changes in matrix compositions can be challenging especially with time-resolved fluorescence resonance energy transfer (FRET). The present disclosure provides these and other needs.

### BRIEF SUMMARY

In one embodiment, the disclosure provides a method for determining an unknown concentration of hematocrit (% HCT) in a test sample having an analyte contained therein, the method comprising:
a) adding a uniform volume or concentration of an analyte to a sample;
b) adding a known amount of a light emitting material to the sample, wherein the light emitting material produces a light output;
c) determining an algorithmic relationship between the light output versus percent hematocrit in the sample using at least two known different hematocrit concentration levels in the sample; and
d) determining an unknown concentration of (% HCT) hematocrit using the measured light output from the light emitting material and the algorithmic relationship determined in step c in the test sample having the analyte.

An algorithmic relationship can be, for example, a linear, a non-linear, a logarithmic, an exponential or polynomial curve fitting algorithm.

In certain aspects, the analyte is an anti-TNFα drug, a protein, a vitamin or an inflammatory protein.

In another embodiment, the disclosure provides a method for determining an analyte plasma concentration within whole blood in a test sample, the method comprising:
a) adding a uniform volume or concentration of an analyte to a sample;
b) adding a known amount of a light emitting material to the sample, wherein the light emitting material produces a light output;
c) measuring at least two distinct light outputs from the light emitting material, the first light output correlates to a known amount of the light emitting material and the second light output is used to determine the analyte concentration;
d) determining an algorithmic relationship between the output of the known amount of the light emitting material and a known % hematocrit concentration;
e) determining the hematocrit concentration in a test sample using the algorithmic relationship in step d;
f) determining a mathematical relationship between a calibration curve for hematocrit and the analyte signal output; and
g) adjusting either the calibration curve or the output from the calibration curve to determining the analyte plasma concentration in the test sample by accounting for the amount of hematocrit within the sample in accordance with steps e and f.

In certain aspects, the analyte is an anti-TNFα drug, protein, vitamin or an inflammatory protein such as C-reactive protein.

In another embodiment, the disclosure provides a method for determining the amount of a buffer added to a test sample, the method comprising:
a) adding a uniform volume or concentration of an analyte to a sample;
b) adding a known amount of a light emitting material to the sample, wherein the light emitting material produces a light output;
c) measuring at least two distinct light outputs in the sample, the first light output correlates to a known amount of light emitting material and the second light output is used to determine the analyte concentration;
d) determining an algorithmic relationship between the output of the known amount of the light emitting material and the buffer volume added to the sample; and
e) determining the volume of buffer added to a test sample using the algorithmic relationship.

In certain aspects, the analyte is fecal calprotectin.

In another embodiment, the disclosure provides a method for determining an analyte concentration using a FRET assay having a donor and an acceptor in an unknown buffer concentration in a test sample, the method comprising:
a) adding a uniform volume or concentration of an analyte to a sample;
b) adding a known amount of a light emitting material to the sample, wherein the light emitting material produces a light output;
c) measuring at least two distinct light outputs in the sample, the first light output is correlated to a known amount of light emitting material and the second light output is used to determine the analyte concentration;
d) determining an algorithmic relationship between the output of the known amount of light emitting material and a buffer volume added to the sample;
e) determining the buffer volume added to the test sample;
f) determining an algorithmic relationship between the buffer volume added and the analyte signal output; and
g) adjusting either the calibration curve or the output from a calibration curve to determining the analyte plasma concentration by accounting for the buffer volume added within the test sample in accordance with steps e and f.

In still yet another embodiment, the disclosure provides a method for determining a correction factor for an assay of a sample. The method comprises adding a selected amount of a light emitting material to the sample. The method further comprises obtaining a measurement of an observed light output from the light emitting material within the sample. The method further comprises applying an algorithm relating the observed light output measurement and the expected light output from the sample to determine the correction factor.

In some embodiments, the applying of the algorithm comprises calculating the correction factor using a function of the observed light output measurement and the selected amount of the light emitting material or the expected light output. In some embodiments, the applying of the algorithm comprises calculating the correction factor using a function of the observed light output measurement and the selected amount of the light emitting material or expected light output. In some embodiments, the applying of the algorithm comprises retrieving a value from a lookup table. In some embodiments, the algorithm is derived from previous measurements using the assay. In certain aspects, the previous measurements are of observed light output from two or more previous samples, wherein at least two of the two or more previous samples have different matrices from one another. In certain aspects, the previous measurements are of observed light output from two or more previous samples, wherein at least two of the two or more previous samples have different volumes from one another.

In some embodiments, the sample is a first sample, and the method further comprises recording a second measurement of an observed light output from a second sample using the assay, and adjusting the second measurement using the determined correction factor, thereby calculating a corrected measurement. In certain aspects, the second sample has the same matrix as the first sample. In certain aspects, the second sample has the same volume as the first sample.

In some embodiments, the light output comprises fluorescence light emitted from the sample. In certain aspects, the sample comprises a FRET system. In certain aspects, the light emitting material comprises a lanthanide fluorophore. In certain aspects, the lanthanide fluorophore comprises a cryptate. In some embodiments, the light output comprises chemiluminescence light emitted from the sample.

In some embodiments, the sample comprises red blood cells. In some embodiments, the concentration factor is used to normalize hematocrit levels in the sample.

These and other embodiments, aspects and objects will become more apparent when read with the detailed description and figures which follow.

### BRIEF DESCRIPTION OF THE DRAWINGS

FIG. 1 is a flowchart of a process in accordance with an embodiment.

FIG. 2 is a graph of FRET donor fluorescence signal versus hematocrit level in various samples.

FIG. 3 is a graph of fluorescence versus matrix volume in various samples.

FIG. 4 is a graph of fluorescence versus % HCT.

FIG. 5 is a graph of fluorescence versus 1/volume.

FIG. 6 is a graph of % error versus buffer volume.

### DETAILED DESCRIPTION

The present disclosure generally relates to methods for accommodating matrix effects in samples that are assayed for light output. These techniques provide advantageous properties allowing them to determine the degree to which the characteristics of measured light output from a sample are affected by the sample environment that one or more analytes of interest are present in. For example, it can be beneficial for an operator of an assay to ascertain and correct for any offset in readings from one sample to another that are caused not by changes in the analyte amount or concentration in the samples, but by the amount of one or more other sample constituents. In can also be beneficial for the assay operator to be able to compensate for changes in sample volume among different samples that are analyzed.

The inventors have now discovered that introducing an exogenous light source to the internal environment of a sample can allow for the determination and correction of sample matrix effects. In particular, it has been found that a known amount of light producing material can be added into a sample, e.g., a homogeneous mixture used to measure an analyte. The light detected from the light producing material is then measured, and by comparing the expected known signal to the actual measured signal from the sample, one can mathematically adjust the assay output to accommodate for one or both of sample matrix volume variations and sample matrix composition variations. The methods provided herein can be used for all assays that measure light, including luminescence assays, chemiluminescence assays, fluorescence assays, and absorbance assays. Advantageously, the disclosed methods can allow for a more robust assay, and can enable a higher tolerance for assay procedure differences. The provided methods also provide the additional benefit of allowing one to identify or estimate sample properties, e.g., sample volumes or sample matrix compositions, through comparisons of predicted and observed light output measurements.

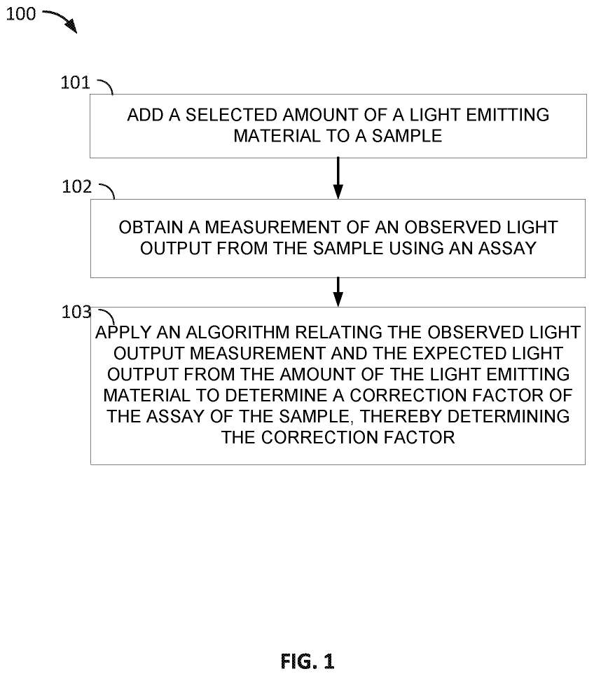

FIG. 1 presents a flowchart of a method (100) in accordance with an embodiment for determining a correction factor for an assay of a sample. In operation 101, a selected amount of a light emitting material such as a specific concentration is added to the sample. In operation 102, a measurement of an observed light output from the sample is obtained using the assay. In operation 103, an algorithm is applied to determine the correction factor, wherein the algorithm relates the observed light output measurement and the expected light output from the amount of the light emitting material to determine the correction factor for the assay of the sample. The algorithm can be a linear, a non-linear, a logarithmic, an exponential or polynomial curve fitting algorithm, wherein the applying of the algorithm can be adjusting a calibration curve to adjust the output concentration. In certain instances, the calibration curve has been previously prepared using known amounts of analyte and therefore, the expected light output is known from previous measurements.

The assay of the provided method can generally be any assay involving the measurement of light output from a sample. The assay can include, for example, one or more of fluorometry, spectrophotometry, colorimetry, and spectroscopy. The assay can in general be used to measure one or more of light emission, light transmission, light absorbance, and light reflection.

Various assay instruments and devices are suitable for use with the methods disclosed herein. For example, a spectrophotometer can be used to measure fluorescence emission light. Fluorescence is the molecular absorption of light energy at one wavelength and its nearly instantaneous re-emission at another, longer wavelength. Some molecules fluoresce naturally, and others must be modified to fluoresce. Compensation for differences and changes in matrix compositions can be challenging especially with time-resolved fluorescence resonance energy transfer (FRET) is used. The methods herein are particularly suitable for time-resolved fluorescence resonance energy transfer (FRET).

A fluorescence spectrophotometer or fluorometer, fluorospectrometer, or fluorescence spectrometer measures the fluorescence light emitted from a sample at different wavelengths, after illumination with light source such as a xenon flash lamp. Fluorometers can have different channels for measuring differently colored fluorescence signals (that differ in their wavelengths), such as green and blue, or ultraviolet and blue, channels. In one aspect, a suitable assay device includes an ability to perform a time-resolved fluorescence resonance energy transfer (FRET) experiment.

In one embodiment, the disclosure provides a method for determining an unknown concentration of hematocrit (% HCT) in a test sample having an analyte contained therein, the method comprising:
a) adding a uniform volume or concentration of an analyte to a sample;
b) adding a known amount of a light emitting material to the sample, wherein the light emitting material produces a light output;
c) determining an algorithmic relationship between the light output versus percent hematocrit in the sample using at least two known different hematocrit concentration levels in the sample; and
d) determining an unknown concentration of (% HCT) hematocrit using the measured light output from the light emitting material and the algorithmic relationship determined in step c in the test sample having the analyte.

In another embodiment, the disclosure provides a method for determining an analyte plasma concentration within whole blood in a test sample, the method comprising:
a) adding a uniform volume or concentration of an analyte to a sample;
b) adding a known amount of a light emitting material to the sample, wherein the light emitting material produces a light output;
c) measuring at least two distinct light outputs from the light emitting material, the first light output correlates to a known amount of light emitting material and the second light output is used to determine the analyte concentration;
d) determining an algorithmic relationship between the output of the known amount of the light emitting material and a known % hematocrit concentration;
e) determining the hematocrit concentration in the test sample using the algorithmic relationship in step d;
f) determining a mathematical relationship between a calibration curve for hematocrit and the analyte signal output; and
g) adjusting either the calibration curve or the output from the calibration curve to determining the analyte plasma concentration of the analyte in the test sample by accounting for the amount of hematocrit within the sample in accordance with steps e and f.

In still another embodiment, the disclosure provides a method for determining the amount of a buffer added to a test sample, the method comprising:
a) adding a uniform volume or concentration of an analyte to a sample;
b) adding a known amount of a light emitting material to the sample, wherein the light emitting material produces a light output;
c) measuring at least two distinct light outputs in the sample, the first light output correlates to a known amount of light emitting material and the second light output is used to determine the analyte concentration;
d) determining an algorithmic relationship between the output of the known amount of light emitting material and the buffer volume added to the sample; and
e) determining the volume of buffer added to a test sample using the algorithmic relationship.

In still yet another embodiment, the disclosure provides a method for determining an analyte concertation using a FRET assay having a donor and an acceptor in an unknown buffer concentration in a test sample, the method comprising:
a) adding a uniform volume or concentration of an analyte to a sample;
b) adding a known amount of light emitting material to the sample, wherein the light emitting material produces a light output;
c) measuring at least two distinct light outputs in the sample, the first light output is correlated to a known amount of light emitting material and the second light output is used to determine the analyte concentration;
d) determining an algorithmic relationship between the output of the known amount of light emitting material and a buffer volume added to the sample;
e) determining the buffer volume added to the test sample;
f) determining an algorithmic relationship between the buffer volume added and the analyte signal output; and
g) adjusting either the calibration curve or the output from a calibration curve to determining the analyte plasma concentration by accounting for the buffer volume added within the sample in accordance with steps e and f.

In certain aspects, the algorithmic relationship and/or the mathematical relationship are each independently a member selected from the group of a linear, a non-linear, a logarithmic, an exponential or polynomial curve fitting algorithm. The algorithmic relationship or mathematical relationship can be a linear regression to produce a straight line that corresponds to y=mx+b.

In certain aspects, the analyte is an endogenous component or an exogenous component found in the blood of a subject The subject can be a mammal such as a human. In one aspect, the analyte is an anti-TNFα drug, a protein, a vitamin or an inflammatory protein.

In one aspect, the anti-TNFα drug is a member selected from the group consisting of REMICADE™ (infliximab), INFLECTRA (Infliximab-dyyb), RENFLEXIS (Infliximab-abda), FLIXABI (Infliximab Biosimilar), REMSIMA (Infliximab Biosimilar), ENBREL™ (etanercept), HUMIRA™ (adalimumab), AMJEVITA (Adalimumab-atto), IMRALDI (Adalimumab Biosimilar), CYLTEZO (Adalimumab Biosimilar), HYRIMOZ (Adalimumab Biosimilar), HULIO (Adalimumab Biosimilar), CIMZIA® (certolizumab pegol), and combinations thereof.

In certain aspects, the anti-TNFα drug is REMICADE™ (infliximab).

In certain aspects, the analyte is C-reactive protein (CRP).

In certain aspects, “a sample” is a known sample having a known concentration or volume of for example, an analyte or buffer volume. A “test sample” is an unknown sample having an unknown amount of for example, an analyte or buffer volume.

In certain aspects, the amount of analyte such as anti-TNFα drug added to a sample or in the sample is between 0 μg and 1000 μg. For example, the amount can be approximately 0 μg, 25 μg, 50 μg, 75 μg, 100 μg, 125 μg, 150 μg, 175 μg, 200 μg, 225 μg, 250 μg, 275 μg, 300 μg, 325 μg, 350 μg, 375 μg, 400 μg, 425 μg, 450 μg, 475 μg, 500 μg, 525 μg, 550 μg, 575 μg, 600 μg, 625 μg, 650 μg, 675 μg, 700 μg, 725 μg, 750 μg, 775 μg, 800 μg, 825 μg, 850 μg, 875 μg, 900 μg, 925 μg, 950 μg, 975 μg, and/or 1000 μg. In certain aspects, the amount of analyte in the sample is approximately 0 μg, 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, and/or 50 μg.

In certain aspects, the analyte is at least two known different hematocrit concentration levels are selected from (i) 1-15% and (ii) 16-75%. In other aspects, the at least two different concentrations are any two values selected from the following percentages: 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, and/or 75%. The two values maybe fractions of the foregoing percentages.

In certain aspects, the concentration of the light emitting material in the sample can range, for example, from 1 fM to 1 mM, e.g., from 1 fM to 16 nM, from 16 fM to 250 nM, from 250 fM to 4 μM, from 4 pM to 63 μM, or from 63 pM to 1 mM. In terms of upper limits, the light emitting material concentration can be less than 1 mM, e.g., less than 63 μM, less than 4 μM, less than 250 nM, less than 16 nM, less than 1 nM, less than 63 pM, less than 4 pM, less than 250 fM, or less than 16 fM. In terms of lower limits, the light emitting material concentration can be greater than 1 fM, e.g., greater than 16 fM, greater than 250 fM, greater than 4 pM, greater than 63 pM, greater than 1 nM, greater than 16 nM, greater than 250 nM, greater than 4 μM, or greater than 63 μM. Higher concentrations, e.g., greater than 1 mM, and lower concentrations, e.g., less than 1 fM, are also contemplated.

In some aspect, the normal concentration of C-reactive protein in the blood is below 3 mg/L. In some embodiments, an elevated concentration of C-reactive protein in the blood is at least 15 mg/L. In certain embodiments, an elevated concentration of C-reactive protein in the blood is at least 30 mg/L. The amount of analyte such as C-reactive protein added to a sample is between 0 μg and 100 μg such as approximately 0 μg, 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, and/or 100 pg.

In some embodiments, the analyte is vitamin D. The normal concentration of vitamin D in the blood is about 20 ng/mL to about 50 ng/mL (e.g., about 20 ng/mL, 23 ng/mL, 25 ng/mL, 27 ng/mL, 29 ng/mL, 31 ng/mL, 33 ng/mL, 35 ng/mL, 37 ng/mL, 39 ng/mL, 41 ng/mL, 43 ng/mL, 45 ng/mL, 47 ng/mL, 49 ng/mL, or 50 ng/mL). These amounts can be used to generate a standard curve.

In some embodiments, an elevated concentration of vitamin D in the blood is at least 50 ng/mL (e.g., 60 ng/mL, 70 ng/mL, 80 ng/mL, 90 ng/mL, 100 ng/mL, 110 ng/mL, 120 ng/mL, 130 ng/mL, 140 ng/mL, 150 ng/mL, 160 ng/mL, 170 ng/mL, 180 ng/mL, 190 ng/mL, 200 ng/mL, 210 ng/mL, 220 ng/mL, 230 ng/mL, 240 ng/mL, 250 ng/mL, 260 ng/mL, 270 ng/mL, 280 ng/mL, 290 ng/mL, 300 ng/mL, 310 ng/mL, 320 ng/mL, 330 ng/mL, 340 ng/mL, 350 ng/mL, 360 ng/mL, 370 ng/mL, 380 ng/mL, 390 ng/mL, 400 ng/mL, 410 ng/mL, 420 ng/mL, 430 ng/mL, 440 ng/mL, 450 ng/mL, 460 ng/mL, 470 ng/mL, 480 ng/mL, 490 ng/mL, or 500 ng/mL). These amounts can be used to generate a standard curve.

In some embodiments, an elevated concentration of vitamin D in the blood is at least 100 ng/mL (e.g., at least 110 ng/mL, 120 ng/mL, 130 ng/mL, 140 ng/mL, 150 ng/mL, 160 ng/mL, 170 ng/mL, 180 ng/mL, 190 ng/mL, 200 ng/mL, 210 ng/mL, 220 ng/mL, 230 ng/mL, 240 ng/mL, 250 ng/mL, 260 ng/mL, 270 ng/mL, 280 ng/mL, 290 ng/mL, 300 ng/mL, 310 ng/mL, 320 ng/mL, 330 ng/mL, 340 ng/mL, 350 ng/mL, 360 ng/mL, 370 ng/mL, 380 ng/mL, 390 ng/mL, 400 ng/mL, 410 ng/mL, 420 ng/mL, 430 ng/mL, 440 ng/mL, 450 ng/mL, 460 ng/mL, 470 ng/mL, 480 ng/mL, 490 ng/mL, or 500 ng/mL). These amounts can be used to generate a standard curve.

Fecal calprotectin is useful in differentiating between IBD (Inflammatory Bowel Disease) and IBS (Irritable Bowel Syndrome). Typically, IBD (e.g. Crohn's Disease (CD) or Ulcerative Colitis (UC)) has accompanying inflammation whereas IBS does not have inflammation. A higher than normal level of calprotectin indicates inflammation and thus can be used to differentiate between IBD and IBS.

In certain aspects, the concertation amount of calprotectin is in a range of about 10 μg/g to about 800 μg/g (μg per gram of stool). In certain aspects, the range is about 10 μg/g to about 60 μg/g such as about 0, 5, 10, 15, 20, 25, 30, 35, 40, 45, 50, 55, and/or 60 μg/g. In certain aspects, a range of about 10 μg/g to about 60 μg/g is considered normal or healthy. These amounts can be used to generate a standard curve.

In other instances, the concentration amount of calprotectin is in a range of about 10 μg/g to about 100 μg/g, such as 10, 15, 20, 25, 30, 35, 40, 45, 50, 55, 60, 65, 70, 75, 80, 85, 90, 95, and/or 100, which is considered normal or healthy. These amounts can be used to generate a standard curve.

In certain instances, a number about 60 μg/g or about 100 μg/g is considered elevated and abnormal (pg per gram of stool). A concentration of calprotectin in a range of about 100 μg/g to about 800 μg/g, such as 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, and/or 800 is considered abnormal. These amounts can be used to generate a standard curve.

In certain aspects, the methods described herein are used to measure and/or detect VCAM-1. In certain aspects, the concentration or level of VCAM-1 is measured. In certain aspects, the biological sample in which VCAM-1 is measured is whole blood.

In certain aspects, the normal control concentration of VCAM-1 or reference value is about 100 to about 500 ng/mL. In certain aspect, the normal amount of VCAM-1 is about 100 ng/mL, 110 ng/mL, 120 ng/mL, 130 ng/mL, 140 ng/mL, 150 ng/mL, 160 ng/mL, 170 ng/mL, 180 ng/mL, 190 ng/mL, 200 ng/mL, 210 ng/mL, 220 ng/mL, 230 ng/mL, 240 ng/mL, 250 ng/mL, 260 ng/mL, 270 ng/mL, 280 ng/mL, 290 ng/mL, 300 ng/mL, 310 ng/mL, 320 ng/mL, 330 ng/mL, 340 ng/mL, 350 ng/mL, 360 ng/mL, 370 ng/mL, 380 ng/mL, 390 ng/mL, 400 ng/mL, 410 ng/mL, 420 ng/mL, 430 ng/mL, 440 ng/mL, 450 ng/mL, 460 ng/mL, 470 ng/mL, 480 ng/mL, 490 ng/mL, and 500 ng/mL. These amounts can be used to generate a standard curve.

In certain aspects, wherein the % HCT in the unknown test sample is between 10% and 75% in the test sample.

Suitable fluorometers and other assay instruments can hold samples in different ways, including with the use of cuvettes, capillaries, Petri dishes, or microplates. The methods described herein can be performed with any of these sample configurations. In certain aspects, the assay has an optional microplate reader, allowing fluorescence light emission scans in up to 384-well plates. Other suitable assay techniques hold samples in place using surface tension.

In certain aspects, the assay uses a device as disclosed in International Patent Application PCT/IB2019/051213, filed Feb. 14, 2019 and published as WO2019159109, which is hereby incorporated in its entirety by reference for all purposes. The analyzers disclosed therein can be used, for example, in point-of-care (POC) settings to measure the absorbance and fluorescence of a sample with minimal or no user handling or interaction. The disclosed analyzers provide advantageous features of more rapid and reliable analyses of samples having properties that can be detected with each of these two approaches. For example, it can be beneficial to quantify both the fluorescence and absorbance of a blood sample being subjected to a diagnostic assay. In some analytical workflows, the hematocrit of a blood sample can be quantified with an absorbance assay, while the signal intensities measured in a FRET assay can provide information regarding other components of the blood sample.

The apparatus disclosed in International Patent Application PCT/IB2019/051213 is also suitable for use with the provided methods, and can be employed for detecting both an emission light from a sample, and absorbance of a transillumination light by the sample. The application, which is hereby incorporated in its entirety by reference for all purposes, describes a device that comprises a first light source configured to emit an excitation light having an excitation wavelength. The apparatus further comprises a second light source configured to transilluminate the sample with the transillumination light. The apparatus further comprises a first light detector configured to detect the excitation light, and a second light detector configured to detect the emission light and the transillumination light. The apparatus further comprises a dichroic mirror configured to (1) epi-illuminate the sample by reflecting at least a portion of the excitation light, (2) transmit at least a portion of the excitation light to the first light detector, (3) transmit at least a portion of the emission light to the second light detector, and (4) transmit at least a portion of the transillumination light to the second light detector.

Suitable cuvettes for use in the assay of the provided method are disclosed in International Patent Application PCT/IB2019/051215, filed Feb. 14, 2019, published as (WO2019159111) and incorporated herein in its entirety by reference for all purposes. One of the provided cuvettes comprises a hollow body enclosing an inner chamber having an open chamber top. The cuvette further comprises a lower lid having an inner wall, an outer wall, an open lid top, and an open lid bottom. At least a portion of the lower lid is configured to fit inside the inner chamber proximate to the open chamber top. The lower lid comprises one or more (e.g., two or more) containers connected to the inner wall, wherein each of the containers has an open container top. In certain aspects, the lower lid comprises two or more such containers. The lower lid further comprises a securing means connected to the hollow body. The cuvette further comprises an upper lid wherein at least a portion of the upper lid is configured to fit inside the lower lid proximate to the open lid top.

The sample of the provided method can be any sample capable of being analyzed with a light measuring assay as described above. In general, at least a portion of the sample comprises a liquid component, solution, or suspension into which the light emitting material is introduced. Preferably, the sample has a substantially homogeneous composition.

In some embodiments, the sample is a biological sample. Suitable biological samples include, but are not limited to, whole blood, plasma, serum, blood cells, cell samples, urine, spinal fluid, sweat, tear fluid, saliva, skin, mucous membrane, and hair. In certain aspects, whole blood, plasma, serum, blood cells and such are preferred, and whole blood, blood cells, and such are particularly preferred. Whole blood includes samples of whole blood-derived blood cell fractions admixed with plasma. With regard to these whole blood samples, the samples can be subjected to pretreatments such as hemolysis, separation, dilution, concentration, and purification.

In some embodiments, the biological sample is a whole blood or a serum sample. In some embodiments, the sample includes red blood cells. In certain aspects, the red blood cells are derived from whole blood. In certain aspects, the red blood cells are lysed. In other aspects, the sample does not include red blood cells. In some embodiments, the blood sample is treated to lyse the red blood cells. This can be done by diluting a blood sample in a lysing agent, such as deionized distilled water, for example at a concentration of 1:1 (i.e., 1 part blood to 1 part lysing agent or distilled deionized water). Alternatively, the sample can be frozen to lyse the cells.

In certain aspects, the blood sample is diluted after lysis. The blood sample may be diluted 1:10 (i.e., one part sample in 10 parts diluent), 1:500, 1:1000, 1:200, 1:2500, 1:8000 or more. In certain aspects, the sample is diluted 1:2000, i.e., one part blood sample in 2000 parts diluent. In certain aspects, the diluent can be 0.1% trifluoroacetic acid in distilled deionized water, or distilled deionized water. In some embodiments, the blood sample is not processed between lysis and dilution.

In general, the sample can include one or more analytes of interest, wherein at least one of the analytes can be directly or indirectly detected using the assay of the method. The sample further includes a sample matrix, which is defined herein as including all components of a sample other than any analytes of interest. In some embodiments, the sample matrix is an aqueous solution. In some embodiments, the sample matrix includes one or more salts, one or more buffers, one or more assay reagents, or a combination thereof. In some embodiments, the sample matrix is an aqueous buffer solution suitable for stabilizing and storing the one or more analytes. For example, the liquid can have a pH and/or an osmolarity suitable for stabilizing and storing the analytes. The sample matrix can include one or more diluents.

In certain aspects, the disclosure provides a method for determining the amount of a buffer added to a test sample. In certain aspects, the buffer is selected from the group consisting of a citrate buffer, a phosphate buffer, an acetate buffer, or a citrate-phosphate buffer.

In certain aspects, the algorithmic relationship between the output of the known amount of light emitting material and the buffer volume added to the sample is linear. In certain aspects, the output of the known amount of light emitting material is correlated (e.g., proportional) to the buffer volume or is a function of buffer volume. It is possible to determine the volume of buffer added to the test sample by using the light output for the sample and the algorithmic relationship. Optionally, the method further comprises determining the linear regression of the percent error and buffer volume using a five parameter logistic regression.

The volume of the sample can range, for example, from 1 μL to 100 mL, e.g., from 1 μL to 1 mL, from 3.2 μL to 3.2 mL, from 10 μL to 10 mL, from 32 μL to 32 mL, or from 100 μL to 100 mL. In terms of upper limits, the sample volume can be less than 100 mL, e.g., less than 32 mL, less than 10 mL, less than 3.2 mL, less than 1 mL, less than 320 μL, less than 100 μL, less than 32 μL, less than 10 μL, or less than 3.2 μL. In terms of lower limits, the sample volume can be greater than 1 μL, e.g., greater than 3.2 μL, greater than 10 μL, greater than 32 μL, greater than 100 μL, greater than 320 μL, greater than 1 mL, greater than 3.2 mL, greater than 10 mL, or greater than 32 mL. Larger volumes, e.g., greater than 100 mL, and smaller volumes, e.g., less than 1 μL, are also contemplated.

The light emitting material of the provided method can vary widely, but is generally a material that emits light having a wavelength and intensity suitable for accurate detection by the assay. Preferably, the light emitting material is selected from materials that can be dissolved or suspended in the sample matrix such that the concentration or density of the light emitting material is substantially homogeneous within the sample.

In some embodiments, the light emitting material includes one or more chemiluminescent materials. In some embodiments, as a result of adding a chemiluminescent light emitting material to the sample, the sample emits a light output that includes chemiluminescence light. Chemiluminescence is characterized by the emission of light from a material due to a chemical reaction, e.g., a chemical alteration of a chromogenic substance. Examples of chemiluminescent materials include, without limitation, luciferases (e.g. firefly luciferase and bacterial luciferase; e.g. disclosed in U.S. Pat. No. 4,737,456, incorporated herein in its entirety by reference for all purposes), luciferin, 2,3-dihydrophthalazinediones, malate dehydrogenase, urease, peroxidase such as horseradish peroxidase (HRPO), alkaline phosphatase, beta-galactosidase, glucoamylase, lysozyme, saccharide oxidases (e.g., glucose oxidase, galactose oxidase and glucose-6-phosphate dehydrogenase), heterocyclic oxidases (e.g. uricase and xanthine oxidase), lactoperoxidase, microperoxidase, and the like.

For example, a horseradish-peroxidase detection system can be used with the chromogenic substrate tetramethylbenzidine (TMB), which yields a soluble product in the presence of hydrogen peroxide that is detectable at a wavelength of 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm. A urease detection system can be used with a substrate such as urea-bromocresol purple (Sigma Immunochemicals; St. Louis, Mo.).

In some embodiments, the light emitting material includes one or more fluorescent materials. In some embodiments, as a result of adding a fluorescent light emitting material to the sample, the sample emits a light output that includes fluorescence light. In certain aspects, fluorescence can be characterized by wavelength, intensity, lifetime, polarization or a combination thereof. In certain aspects, the introduction of a time delay between a flash excitation and the measurement of the fluorescence at the emission wavelength allows the discrimination of long lived from short-lived fluorescence and the increase of a signal-to-noise ratio. Examples of fluorescent materials include, without limitation, DAPI, fluorescein, Hoechst 33258, R-phycocyanin, B-phycoerythrin, R-phycoerythrin, rhodamine, Texas red, and lissamine.

In some embodiments, the light emitting material includes one or more FRET systems. FRET (fluorescence resonance energy transfer or Förster resonance energy transfer) refers to a mechanism describing energy transfer between a donor compound such as cryptate and an acceptor compound such as Alexa 647, when the donor and acceptor are in proximity to one another and when they are excited at the excitation wavelength of the donor fluorescent compound. A donor compound, initially in its electronic excited state, can transfer energy to an acceptor fluorophore through non-radiative dipole-dipole coupling. The efficiency of this energy transfer is inversely proportional to the sixth power of the distance between donor and acceptor, making FRET extremely sensitive to small changes in distance. After the energy transfer, the acceptor fluoresces or quenches the excitation. It is known that in order for two fluorescent compounds to be FRET partners, the emission spectrum of the donor fluorescent compound must partially overlap the excitation spectrum of the acceptor compound. The preferred FRET-partner pairs are those for which the value R0 (Forster distance, i.e., the distance at which energy transfer is 50% efficient) is greater than or equal to 30 Å.

The measured light output from a FRET system can be any measurable signal representative of FRET between a donor fluorescent compound and an acceptor compound. A FRET signal can therefore be a variation in the intensity or in the lifetime of luminescence of the donor fluorescent compound or of the acceptor compound when the latter is fluorescent. The FRET signal can be measured in different ways. Measurement of the fluorescence emitted by the donor alone, by the acceptor alone or by the donor and the acceptor, or measurement of the variation in the polarization of the light emitted in the medium by the acceptor as a result of FRET. One can also include measurement of FRET by observing the variation in the lifetime of the donor, which is facilitated by using a donor with a long fluorescence lifetime, such as rare earth complexes (especially on simple equipment like plate readers). Furthermore, the FRET signal can be measured at a precise instant or at regular intervals, making it possible to study its change over time and thereby to investigate the kinetics of the biological process studied.

In certain aspects, the FRET assay is a time-resolved FRET assay. Time resolve FRET relies on the use of specific fluorescent molecules that have the property of emitting over long periods of time (measured in milliseconds) after excitation, when most standard fluorescent dyes (e.g. fluorescein) emit within a few nanoseconds of being excited. As a result, it is possible to, for example, excite a cryptate lanthanide using a pulsed light source (e.g., Xenon flash lamp or pulsed laser), and measure after the excitation pulse.

In some embodiments, the light emitting material of the provided method includes one or more lanthanide fluorophores. The lanthanide fluorophore can be, for example, a cryptate. Cryptates are complexes that include a macrocycle within which a lanthanide ion such as terbium or europium can be tightly embedded or chelated. This cage like structure is useful for collecting irradiated energy and transferring the collected energy to the lanthanide ion. The lanthanide ion can release the energy with a characteristic fluorescence. In certain aspects, the light emitting material includes a FRET energy donor compound that is a cryptate, such as a lanthanide cryptate.

In certain aspects, the cryptate has an absorption wavelength between about 300 nm to about 400 nm, such as about 325 nm to about 375 nm. In certain aspects, cryptate dyes have four fluorescence emission peaks at about 490 nm, about 548 nm, about 587 nm, and 621 nm. Thus, as a donor, the cryptate is compatible with fluorescein-like (green zone) molecules, Cy5, DY-647-like (red zone) acceptors, Allophycocyanin (APC), or Phycoeruythrin (PE) to perform TR-FRET experiments.

In certain aspects, the terbium cryptate molecule “Lumi4-Tb” from Lumiphore, marketed by Cisbio bioassays is used as the cryptate donor. The terbium cryptate “Lumi4-Tb” has the chemical structure below.

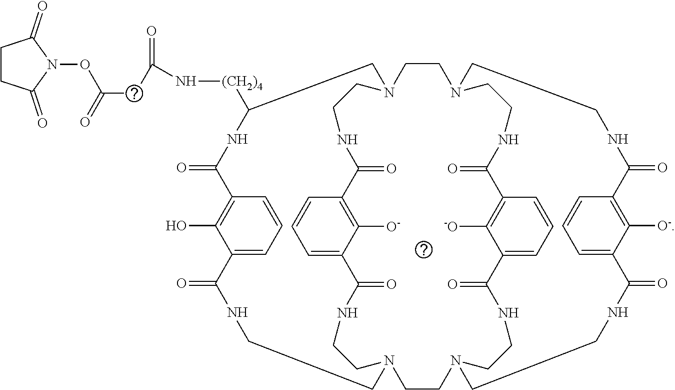

In certain other aspects, cryptates disclosed in International Patent Application Publication WO 2015/157057, which is incorporated herein by reference in its entirety for all purposes, are suitable for use in the present disclosure. This application publication describes cryptate molecules useful for labeling biomolecules. As disclosed therein, certain of the cryptates have a structure as follows:

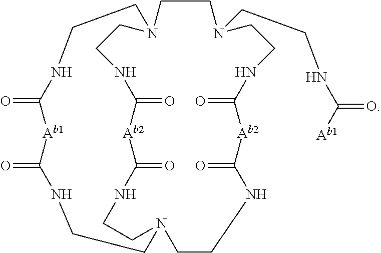

In certain other aspects, a terbium cryptate useful in the present disclosure is shown below:

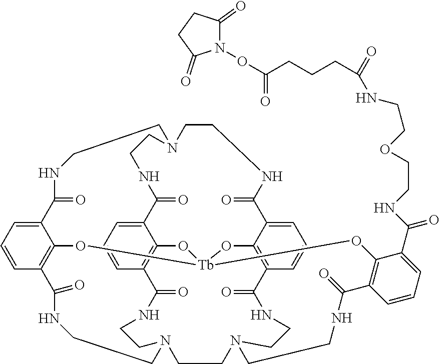

In certain aspects, the cryptates that are useful in the present invention are disclosed in International Patent Application Publication WO 2018/130988, which is incorporated herein by reference in its entirety for all purposes. As disclosed therein, the compounds having the following chemical structure are useful as FRET donors in the present disclosure:

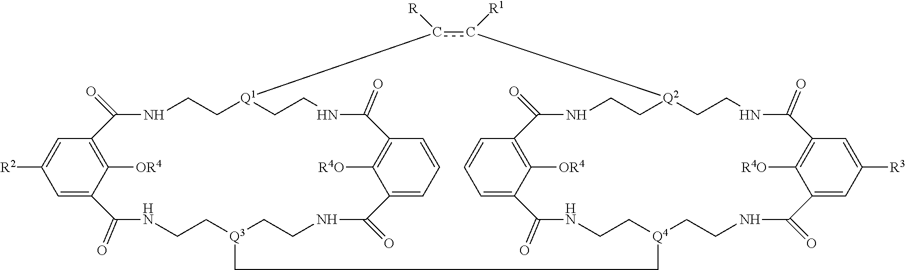

wherein when the dotted line is present, R and R¹ are each independently selected from the group consisting of hydrogen, halogen, hydroxyl, alkyl optionally substituted with one or more halogen atoms, carboxyl, alkoxycarbonyl, amido, sulfonato, alkoxycarbonylalkyl or alkylcarbonylalkoxy or alternatively, R and R¹ join to form an optionally substituted cyclopropyl group wherein the dotted bond is absent; R² and R³ are each independently a member selected from the group consisting of hydrogen, halogen, SO₃H, —SO₂—X, wherein X is a halogen, optionally substituted alkyl, optionally substituted aryl, optionally substituted alkenyl, optionally substituted alkynyl, optionally substituted cycloalkyl, or an activated group that can be linked to a biomolecule, wherein the activated group is a member selected from the group consisting of a halogen, an activated ester, an activated acyl, optionally substituted alkylsulfonate ester, optionally substituted arylsulfonate ester, amino, formyl, glycidyl, halo, haloacetamidyl, haloalkyl, hydrazinyl, imido ester, isocyanato, isothiocyanato, maleimidyl, mercapto, alkynyl, hydroxyl, alkoxy, amino, cyano, carboxyl, alkoxycarbonyl, amido, sulfonato, alkoxycarbonylalkyl, cyclic anhydride, alkoxyalkyl, a water solubilizing group or L; R⁴ are each independently a hydrogen, C₁-C₆ alkyl, or alternatively, 3 of the R⁴ groups are absent and the resulting oxides are chelated to a lanthanide cation; and Q¹-Q⁴ are each independently a member selected from the group of carbon or nitrogen.

In order to detect a FRET signal, a FRET acceptor is required. The FRET acceptor has an excitation wavelength that overlaps with an emission wavelength of the FRET donor. The acceptor molecules that can be used include, but are not limited to, fluorescein-like (green zone) acceptor, Cy5, DY-647, Alexa Fluor 488, Alexa Fluor 546, Allophycocyanin (APC), Phycoeruythrin (PE) and Alexa Fluor 647. Other acceptors include, but are not limited to, cyanin derivatives, D2, CYS, fluorescein, coumarin, rhodamine, carbopyronine, oxazine and its analogs, Alexa Fluor fluorophores, Crystal violet, perylene bisimide fluorophores, squaraine fluorophores, boron dipyrromethene derivatives, NBD (nitrobenzoxadiazole) and its derivatives, and DABCYL (4-((4-(dimethylamino)phenyl)azo)benzoic acid).

In certain aspects of the embodiments, the assay uses a donor fluorophore consisting of terbium bound within a cryptate. The terbium cryptate can be excited with a 365 nm UV LED. The terbium cryptate emits at four (4) wavelengths within the visible region. In one aspect, the assay uses the lowest donor emission energy peak of 620 nm as the donor signal within the assay. In certain aspects, the acceptor fluorophore, when in very close proximity, is excited by the highest energy terbium cryptate emission peak of 490 nm causing light emission at 520 nm. Both the 620 nm and 520 nm emission wavelengths are measured independently in a device or instrument and results can be reported as RFU ratio 620/520. Alternatively, the donor emission or the acceptor emission can be used.

The amount of light emitting material added to the sample is generally known, such that the amount can be a defined quantity in the method algorithm used to determine the correction factor. The amount can be selected based at least in part on factors that can include, for example. expected light occluding properties of the sample, and/or light sensitivity and/or detection limit properties of the assay. The concentration of the light emitting material in the sample can range, for example, from 1 fM to 1 mM, e.g., from 1 fM to 16 nM, from 16 fM to 250 nM, from 250 fM to 4 μM, from 4 pM to 63 μM, or from 63 pM to 1 mM. In terms of upper limits, the light emitting material concentration can be less than 1 mM, e.g., less than 63 μM, less than 4 μM, less than 250 nM, less than 16 nM, less than 1 nM, less than 63 pM, less than 4 pM, less than 250 fM, or less than 16 fM. In terms of lower limits, the light emitting material concentration can be greater than 1 fM, e.g., greater than 16 fM, greater than 250 fM, greater than 4 pM, greater than 63 pM, greater than 1 nM, greater than 16 nM, greater than 250 nM, greater than 4 μM, or greater than 63 μM. Higher concentrations, e.g., greater than 1 mM, and lower concentrations, e.g., less than 1 fM, are also contemplated.

The measurement of light output by the sample can include the measurement of all light output from the sample, or the measurement of light within one or more selected ranges of wavelengths. The measured light can include far infrared light having a wavelength between 15 μm and 1000 μm, e.g., between 15 μm and 930 μm, between 360 μm and 960 μm, between 580 μm and 980 μm, between 730 μm and 990 μm, or between 830 μm and 1000 μm. The measured light can include long-wavelength infrared light having a wavelength between 8 μm and 15 μm, e.g., between 8 μm and 12.2 μm, between 8.7 μm and 12.9 μm, between 9.4 μm and 13.6 μm, between 10.1 μm and 14.3 μm, or between 10.8 μm and 15 μm. The measured light can include mid-wavelength infrared light having a wavelength between 3 μm and 8 μm, e.g., between 3 μm and 6 μm, between 3.5 μm and 6.5 μm, between 4 μm and 7 μm, between 4.5 μm and 7.5 μm, or between 5 μm and 8 μm. The measured light can include short-wavelength infrared light having a wavelength between 1400 nm and 3000 nm, e.g., between 1400 nm and 2400 nm, between 1600 nm and 2500 nm, between 1700 nm and 2700 nm, between 1900 nm and 2800 nm, or between 2000 nm and 3000 nm. The measured light can include near-infrared light having a wavelength between 750 nm and 1400 nm, e.g., between 750 nm and 1100 nm, between 820 nm and 1200 nm, between 880 nm and 1300 nm, between 950 nm and 1300 nm, or between 1000 nm and 1400 nm. The measure light can include visible light having a wavelength between 380 nm and 750 nm, e.g., between 380 nm and 600 nm, between 420 nm and 640 nm, between 450 nm and 680 nm, between 490 nm and 710 nm, or between 530 nm and 750 nm. The measured light can include ultraviolet light having a wavelength between 10 nm and 400 nm, e.g., between 10 nm and 366 nm, between 133 nm and 380 nm, between 218 nm and 389 nm between 278 nm and 396 nm, or between 319 nm and 400 nm. The measured light can include infrared light and visible light. The measured light can include visible light and ultraviolet light. The measured light can include infrared light, visible light, and ultraviolet light.

The algorithm of the method can vary widely, but preferably includes a relationship between the observed light output measurement from the sample, the selected amount of light emitting material added to the sample, and the correction factor for the assay of the sample. In this way, the algorithm can accept the observed light measurement and the known light emitting material amount as inputs to the algorithm, and deliver the correction factor as an output of the algorithm. The expected amount of light output can be a previous measurement using a standard curve. In certain aspects, the algorithm is partially or entirely theoretically derived based on known properties of light and of the components of the sample and the assay instrumentation. In certain aspects, the algorithm is partially or entirely empirically derived based on previous light output measurements from other samples, e.g., reference samples, including the light emitting material.

In some embodiments, the algorithm involves calculating the correction factor using one or a series of mathematical functions relating the observed light output measurement, the selected light emitting material amount, and the correction factor. The one or more functions can express the correction factor in terms of the measured light output and the known amount of light emitting material. The one or more functions can be empirically and/or theoretically derived.

In certain aspects, a function of the algorithm is derived by fitting a curve or line to plotted data points based on earlier measurements. As a non-limiting example, a different known amount of the light emitting material can be added to each of two or more samples having matrices known to not interfere with light output by the light emitting material. The light output from these samples can be measured, and a plot can be constructed of points representing the light emitting material concentration and the light output measurement for the two or more samples. A line or curve, i.e., a standard curve, can then be fit to these data points using any curve fitting technique generally known in the art. The equation of the line or curve can subsequently be used to calculate the expected light output for a known amount of light emitting material added to a future sample having an unknown sample matrix. By comparing the expected light output and a measured light output for this unknown sample, a correction factor can be determined. In certain aspects, the correction factor includes a calculated difference between the expected and measured light outputs. In certain aspects, the correction factor includes a ratio of the expected light output to the measured light output, or vice versa.

In some embodiments, the algorithm involves retrieving a value from a lookup table relating the observed light output measurement, the selected light emitting material amount, and the correction factor. The lookup table can include empirically derived values. In some embodiments, the algorithm includes deriving a calculated value by interpolating among two or more values retrieved from the lookup table. The interpolating can involve any technique generally known in the art.

### EXAMPLES

The present disclosure will be better understood in view of the following non-limiting examples.

### Example 1

In some embodiments, the assay of the provided method is used to measure hematocrit levels in the sample, and the correction factor is used to normalize hematocrit levels in the sample. Hematocrit is the ratio of the volume of packed red blood cells to the total blood volume. It is also known as the packed cell volume, or PCV. Under some conditions there is a linear relationship between hematocrit and the concentration of hemoglobin (ctHb). The relationship can be expressed as follows:

Hct (%)=(0.0485×ctHb (mmol/L)+0.0083)×100

(Kokholm G. Simultaneous measurements of blood pH, pCO2, pO2 and concentrations of hemoglobin and its derivatives—a multicenter study. Radiometer publication AS107. Copenhagen: Radiometer Medical A/S, 1991). It is also known that different amounts of red blood cells in a sample will quench light differently.

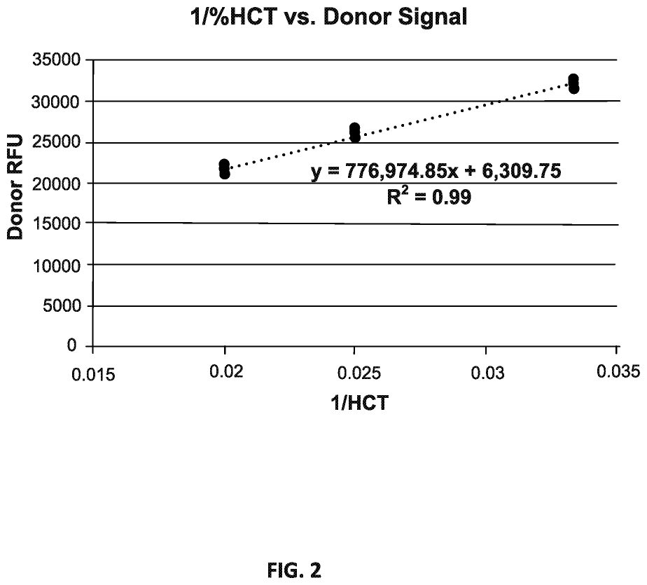

FIG. 2 illustrates a standard curve of Hct (%) samples showing the effect that different amounts of hematocrit have on the fluorescence signal using an identical known amount of light emitting material. Using the equations of the fitted standard curve and the above equation relating hematocrit and hemoglobin concentration, it is possible for one to calculate the total amount of Hb (ctHb) based on a light output measurement. By using the provided methods for deriving a correction factor and adjusting an observed light output measurement, the accuracy of such a hemoglobin concentration determination can be improved.

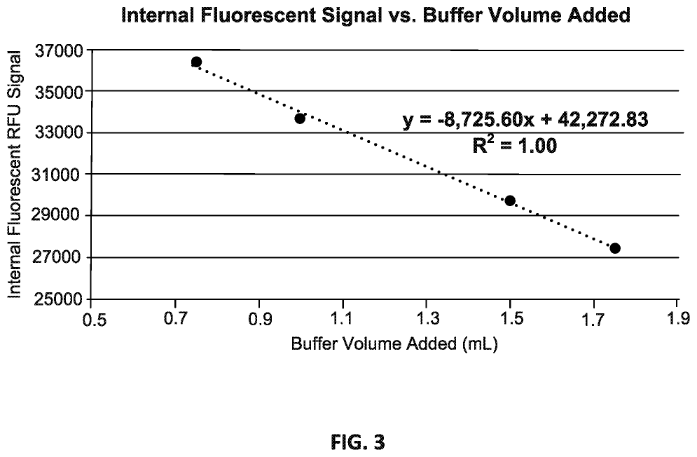

FIG. 3 illustrates a plot of the fluorescence light output signal from four samples to which an identical known amount of light emitting material was added. The samples differ from one another in volume due to various amounts of buffer being added to the sample, diluting the light emitting material to different concentration levels. The results shown in the graph demonstrate the effect that different sample matrix volumes can have on measured light output from the light emitting material within the samples.

### Example 2

This example illustrates a method for determining an unknown infliximab (IFX) concentration within an unknown % HCT sample using a known amount of donor RFU between 0 and 1.56 μg/mL of IFX and % hematocrit (HCT).

The following three tables show the RFU summary data from running a range of IFX levels that span the linear range of the assay. The average of three replicates is shown for the Donor RFU, Acceptor RFU and Acceptor to Donor Ratios at each concentration tested and summarized below in the three Tables, representing 25%, 40% and 53% HCT.

Table 1 summarizing the average Donor RFU

25% HCT

| Avg [IFX] | Donor | Donor |
|---|---|---|
| (μg/mL) | RFU | % CV |
| 50.0 | 94883 | 2% |
| 25.0 | 98653 | 4% |
| 12.5 | 91337 | 22% |
| 6.3 | 107845 | 0% |
| 3.1 | 111902 | 7% |
| 1.6 | 105508 | 5% |
| 0.8 | 93201 | 4% |
| 0.0 | 99728 | 4% |

Table 2 summarizing the average Donor RFU.

40% HCT

| Avg [IFX] | Donor | Donor |
|---|---|---|
| (μg/mL) | RFU | % CV |
| 50.0 | 66266 | 2% |
| 25.0 | 71412 | 4% |
| 12.5 | 72627 | 1% |
| 6.3 | 72466 | 2% |
| 3.1 | 73943 | 1% |
| 1.6 | 74788 | 2% |
| 0.8 | 75627 | 2% |
| 0.0 | 72993 | 4% |

Table 3 summarizing the average Donor RFU for each corresponding IFX concentration.

53% HCT

| Avg [IFX] | Donor | Donor |
|---|---|---|
| (μg/mL) | RFU | % CV |
| 50.0 | 53496 | 1% |
| 25.0 | 53890 | 3% |
| 12.5 | 56222 | 2% |
| 6.3 | 53815 | 1% |
| 3.1 | 55533 | 3% |
| 1.6 | 55011 | 1% |
| 0.8 | 55970 | 1% |
| 0.0 | 55660 | 4% |

A summary of the average Donor RFU for IFX concentrations from 0 to 1.56 μg/mL at each of the three % HCT values are summarized below in Table 4.

| % HCT | Avg Donor RFU (0-1.56 μg/mL) |
|---|---|
| 25 | 99479 |
| 40 | 74469 |
| 53 | 55547 |

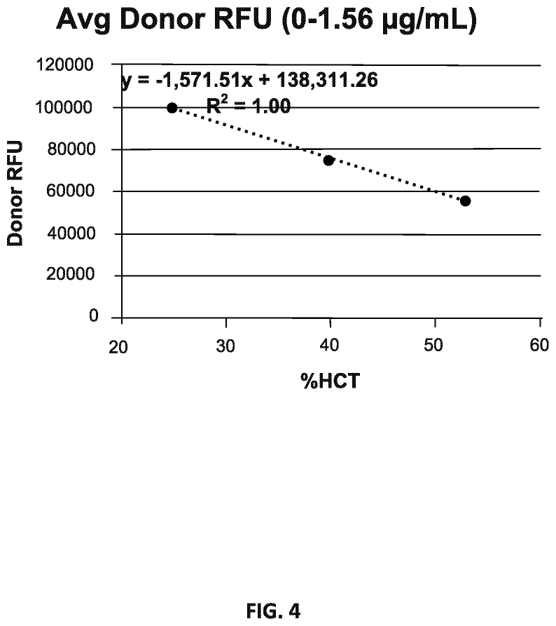

A graph of the values found in Table 4 can be seen in FIG. 4.

It can be seen from FIG. 4 that a linear relationship exists between the Donor RFU and the % HCT tested. This relationship can be used to approximate the % HCT for each sample tested using the measured Donor RFU for each sample and the linear equation (y=mx+b) shown in FIG. 4 (y=−1571.51x+138,311.26). There is no need to run a new standard curve to determine the amount of HCT levels.

A summary of the back calculated % HCT values obtained from using this method can be found in Table 5 below.

| Avg | 25% HCT | | 40% HCT | | 53% HCT | |
|---|---|---|---|---|---|---|
| [IFX] | Avg Calc. | | Avg Calc. | | Avg Calc. | |
| (μg/mL) | % HCT | % CV | % HCT | % CV | % HCT | % CV |
| 50.0 | 28 | 4% | 46 | 1% | 54 | 0% |
| | 25 | 9% | 43 | 5% | 54 | 2% |
| 12.5 | 30 | 42% | 42 | 1% | 52 | 1% |
| 6.3 | 19 | 1% | 42 | 2% | 54 | 1% |
| 3.1 | 17 | 29% | 41 | 1% | 53 | 2% |
| 1.6 | 21 | 17% | 40 | 2% | 53 | 1% |
| 0.8 | 29 | 8% | 40 | 2% | 52 | 1% |
| 0.0 | 25 | 12% | 42 | 5% | 53 | 3% |

This example shows that the amount of hematocrit can be determined from the Donor RFU for each sample and the algorithm of y=mx+b equation shown in FIG. 4. Using a standard curve of infliximab levels, the amount of % HCT can be determined.

### Example 3

This example illustrates a method to determine infliximab (IFX) plasma concentration within whole blood.

It has been discovered that within a given % HCT, the output signal plotted against the infliximab concentration yields a dose response.

Using a single Donor RFU, one can approximate the % HCT of the sample and use it to adjust the IFX result output.

Combining the Donor RFU % HCT determination method with the IFX concentration determination method, the quantitative IFX values are able to be calculated. The quantitative summary results for the 25, 40 and 53% HCT levels tested are shown below in Table 6 below.

| | 25% HCT | | | 40% HCT | | | 53% HCT | | |
|---|---|---|---|---|---|---|---|---|---|
| | Avg | | | Avg | | | Avg | | |
| Known Values | Calc.[IFX] | | % | Calc.[IFX] | | % | Calc.[IFX] | | % |
| [IFX] (μg/mL) | (μg/mL) | % CV | Error | (μg/mL) | % CV | Error | (μg/mL) | % CV | Error |
| 25.0 | 22.8 | 5% | −9% | 24.6 | 7% | −2% | 25.3 | 5% | 1% |
| 12.5 | 14.1 | 30% | 13% | 13.0 | 1% | 4% | 12.0 | 4% | −4% |
| 6.3 | 5.7 | 1% | −8% | 6.7 | 1% | 7% | 6.7 | 7% | 7% |
| 3.1 | 2.9 | 7% | −6% | 3.3 | 0% | 6% | 3.2 | 6% | 3% |
| 1.6 | 1.6 | 3% | 3% | 1.6 | 2% | 3% | 1.6 | 10% | 4% |
| 0.8 | 0.9 | 18% | 10% | 0.7 | 16% | −16% | 0.8 | 39% | 7% |
| 0.0 | 0.0 | NA | NA | −0.2 | NA | NA | −0.3 | NA | NA |

This is a summary of the quantitative IFX results obtained at each % HCT calculated by using each samples Donor RFU to approximate the % HCT which is then used to calculate the individual sample result.

The method described above for determining the IFX concentration within an unknown % HCT sample shows acceptable accuracy and precision when testing at levels of whole blood that span 25-53% HCT.

### Example 4

This example illustrates a method for determining fecal calprotectin (FCP) concentration within an unknown buffer.

Materials in Table 7 below:

| | Part Number/Lot | |
|---|---|---|
| Material | Number | Additional Information |
| FCP Assay Calibrators | PPN 2938 | Prepared in 1X TBS with 0.1% BSA |
| Acceptor/Donor | 041019 | Prepared in 1X TBS, 0.1% BSA, and |
| Conjugate (Liquid) | | 0.05% Proclin150 |
| Assay Buffer | PPN 4403 | 1X TBS with 0.1% PVP |
| FRET Reaction Vessel | PPN 4306, Lot | N/A |
| | #16325 | |

The RFU summary data from running a range of FCP levels that span the linear range of the assay. Each concentration was run in singlet at 0.75, 0.875, 1.0, 1.25, and 1.5 mL. The results are summarized below in Table 8 for each volume and concentration tested.

| | | Donor |
|---|---|---|
| Volume | μg/g | RFU |
| 0.750 | 1733 | 308117 |
| mL | 1300 | 318979 |
| | 433 | 346150 |
| | 35 | 361034 |
| | 0 | 362165 |
| 0.875 | 1733 | 267888 |
| mL | 1300 | 274690 |
| | 433 | 293861 |
| | 35 | 318780 |
| | 0 | 312885 |
| 1.000 | 1733 | 239353 |
| mL | 1300 | 244124 |
| | 433 | 271218 |
| | 35 | 275226 |
| | 0 | 278964 |
| 1.250 | 1733 | 195278 |
| mL | 1300 | 197052 |
| | 433 | 212697 |
| | 35 | 219888 |
| | 0 | 222949 |
| 1.500 | 1733 | 168390 |
| mL | 1300 | 165250 |
| | 433 | 173303 |
| | 35 | 185286 |
| | 0 | 186599 |

Table 8 summarizes the Donor RFU, Acceptor RFU and Acceptor to Donor Ratio for each corresponding FCP concentration for each buffer volume tested at 5 min.

FCP Determination

Donor RFU Buffer Volume Determination Method

It was found that plotting 1/Volume (mL) vs. observed Donor RFU yielded a strong correlation. A table summarizing the average Donor RFU values at different buffer volume concentrations tested at 5 minutes is found below Table 9. The Donor RFU values are taken from the average Donor RFU observed at concentrations of 0, 35 and 433 μg/g of FCP.

| Buffer Volume (mL) | Donor RFU |
|---|---|
| 0.75 | 356450 |
| 0.875 | 308509 |
| 1 | 275136 |
| 1.25 | 218511 |
| 1.5 | 181729 |

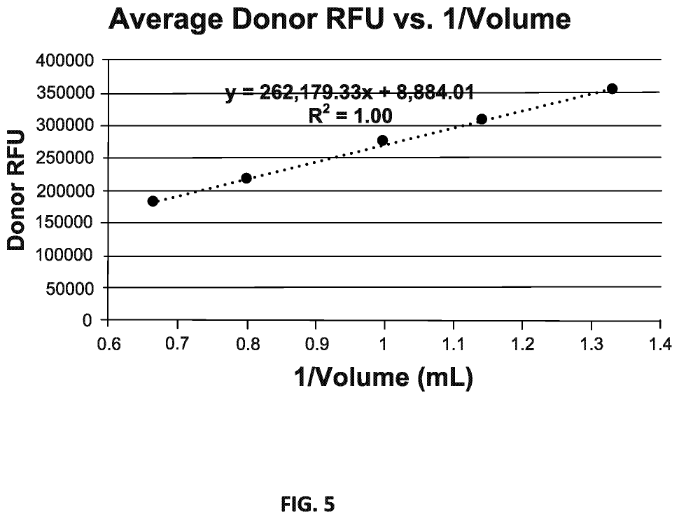

A graph of the values found in Table 9 can be seen in FIG. 5, which figure shows the average Donor RFU between 0 and 6.25 μg/mL FCP vs. 1/Volume.

It can be seen from FIG. 5 that a linear relationship exists between the Donor RFU and 1/Volume at the volumes tested. This relationship can be used to approximate the volume of buffer added for each sample tested using the measured Donor RFU for each sample and the y=mx+b equation shown in FIG. 5.

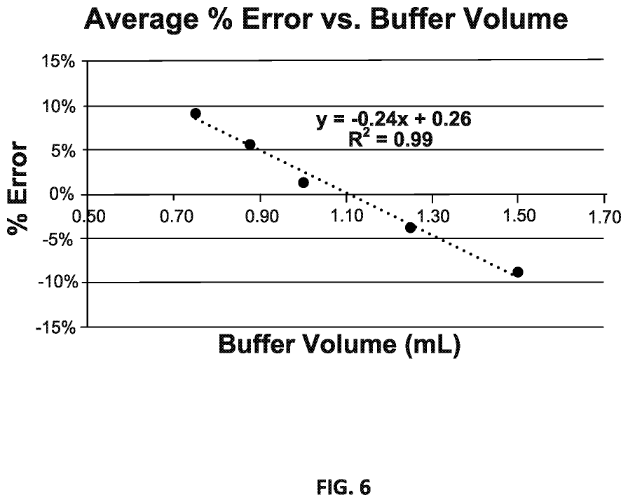

Using the calculated volume determined from the Donor RFU using the equation found in FIG. 5 it is possible to determine the percent offset in the quantitative value from using a single 5-PL calibration curve. This percent error offset can then be used to adjust the output from the original concentration obtained from the 5-PL calibration curve. FIG. 6 shows the linear regression plots of plotting the average percent error across the calibration range vs. buffer volume when using a single 5-PL calibration curve.

Using a 1.0 mL buffer volume standard curve, a 5-PL fit is used to create a calibration curve.

The 5-PL parameters obtained from running a FCP calibration curve using 1.0 mL of buffer are shown:

Formula:

[symbol]

Equation parameters

| Parameter | Description | Value |
|---|---|---|
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |

| | |
|---|---|
| [symbol] | [symbol] |
| [symbol] | [symbol] |

[symbol]

indicates data missing or illegible when filed

Next, using the 5-PL fit for all buffer volumes used during the testing, the concentration is calculated for each level and corresponding buffer volume added. The average percent error is calculated for each buffer volume added. A table summarizing the average percent error for each buffer volume added is found in the Table.

| Volume | Average % |
|---|---|
| (mL) | Error |
| 0.75 | 9% |
| 0.88 | 6% |
| 1.00 | 1% |
| 1.25 | −4% |
| 1.50 | −9% |

Combining the Donor RFU vs. 1/Volume (mL) buffer volume approximation method described with the FCP concentration offset determination method from a single 5-PL calibration curve, the quantitative FCP values are able to be calculated.

The quantitative summary results for known concentrations of FCP tested with buffer volumes of 0.75, 0.875, 1.0, 1.25 and 1.5 mL are shown below in the Table.

| | Final Calculated [fCP] (μg/g) | | | | | | |
|---|---|---|---|---|---|---|---|
| [fCP] | 0.75 | 0.875 | 1.0 | 1.25 | 1.5 | | |
| (μg/g) | mL | mL | mL | mL | mL | Avg Calc. Conc. | % CV |
| 1733 | 1819 | 1805 | 1745 | 1670 | 1619 | 1731 | 5% |
| 1300 | 1313 | 1317 | 1302 | 1293 | 1305 | 1306 | 1% |
| 433 | 434 | 445 | 421 | 435 | 462 | 439 | 3% |
| 35 | 35 | 34 | 36 | 38 | 37 | 36 | 5% |
| 0 | <1 | <1 | <1 | 1 | 1 | NA | NA |

The results are calculated by using each samples Donor RFU to approximate the volume which is then used to adjust the quantitative output from a single 5-PL calibration curve output to yield a result.

The method described in this report for determining the FCP concentration within an unknown buffer volume added shows accuracy and precision when testing at different concentrations of FCP spanning buffer addition volumes of 0.75-1.5 mL.

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A method for determining an unknown concentration of hematocrit (% HCT) in a test sample having an analyte contained therein, the method comprising:
a) adding a uniform volume or concentration of an analyte to a sample;
b) adding a known amount of a light emitting material to the sample, wherein the light emitting material produces a light output;
c) determining an algorithmic relationship between the light output versus percent hematocrit in the sample using at least two known different hematocrit concentration levels in the sample; and
d) determining an unknown concentration of (% HCT) hematocrit using the measured light output from the light emitting material and the algorithmic relationship determined in step c in the test sample having the analyte.

2. The method of claim 1, wherein the analyte is an anti-TNFα drug or an inflammatory protein.

3. The method of claim 1, wherein the anti-TNFα drug is a member selected from the group consisting of REMICADE™ (infliximab), INFLECTRA (Infliximab-dyyb), RENFLEXIS (Infliximab-abda), FLIXABI (Infliximab Biosimilar), REMSIMA (Infliximab Biosimilar), ENBREL™ (etanercept), HUMIRA™ (adalimumab), AMJEVITA (Adalimumab-atto), IMRALDI (Adalimumab Biosimilar), CYLTEZO (Adalimumab Biosimilar), HYRIMOZ (Adalimumab Biosimilar), HULIO (Adalimumab Biosimilar), CIMZIA® (certolizumab pegol), and combinations thereof.

4. The method of claim 3, wherein the anti-TNFα drug is REMICADE™ (infliximab).

5. The method of claim 1, wherein the analyte is C-reactive protein (CRP).

6. The method of claim 1, wherein the at least two known different hematocrit concentration levels are two concentrations selected from (i) 1-15% and (ii) 16-75%.

7. The method of claim 1, wherein the algorithmic relationship is a member selected from the group consisting of a linear, a non-linear, a logarithmic, an exponential or polynomial curve fitting algorithm.

8. The method of claim 7, wherein the algorithmic relationship is a linear curve fitting algorithm.

9. The method of claim 1, wherein the % HCT in the test sample is between 10% and 75% in the test sample.

10. A method for determining an analyte plasma concentration within whole blood in a test sample, the method comprising:
a) adding a uniform volume or concentration of an analyte to a sample;
b) adding a known amount of a light emitting material to the sample, wherein the light emitting material produces a light output;
c) measuring at least two distinct light outputs from the light emitting material, the first light output correlates to a known amount of light emitting material and the second light output is used to determine the analyte concentration;
d) determining an algorithmic relationship between the output of the known amount of light emitting material and a known % hematocrit concentration;
e) determining the hematocrit concentration in the test sample using the algorithmic relationship in step d;
f) determining a mathematical relationship between a calibration curve for hematocrit and analyte signal output; and
g) adjusting either the calibration curve or the output from the calibration curve to determining the analyte plasma concentration of the analyte in the test sample by accounting for the amount of hematocrit within the sample in accordance with steps e and f.

11. The method of claim 10, wherein the analyte is an anti-TNFα drug or an inflammatory protein.

12. The method of claim 10, wherein the anti-TNFα drug is a member selected from the group consisting of REMICADE™ (infliximab), INFLECTRA (Infliximab-dyyb), RENFLEXIS (Infliximab-abda), FLIXABI (Infliximab Biosimilar), REMSIMA (Infliximab Biosimilar), ENBREL™ (etanercept), HUMIRA™ (adalimumab), AMJEVITA (Adalimumab-atto), IMRALDI (Adalimumab Biosimilar), CYLTEZO (Adalimumab Biosimilar), HYRIMOZ (Adalimumab Biosimilar), HULIO (Adalimumab Biosimilar), CIMZIA® (certolizumab pegol), and combinations thereof.

13. The method of claim 12, wherein the anti-TNFα drug is REMICADE™ (infliximab).

14. The method of claim 10, wherein the analyte is C-reactive protein (CRP).

15. The method claim 10, wherein the algorithmic relationship is a member selected from the group consisting of a linear, a non-linear, a logarithmic, an exponential or polynomial curve fitting algorithm.

16. (canceled)

17. (canceled)

18. The method of claim 16, wherein the algorithmic relationship is a member selected from the group consisting of a linear, a non-linear, a logarithmic, an exponential or polynomial curve fitting algorithm.

19. A method for determining an analyte concertation using a FRET assay having a donor and an acceptor in an unknown buffer concentration in a test sample, the method comprising:
a) adding a uniform volume or concentration of an analyte to a sample;
b) adding a known amount of a light emitting material to the sample, wherein the light emitting material produces a light output;
c) measuring at least two distinct light outputs in the sample, the first light output is correlated to a known amount of light emitting material and the second light output is used to determine the analyte concentration;
d) determining an algorithmic relationship between the output of the known amount of light emitting material and the buffer volume added to the sample;
e) determining the buffer volume added to the test sample;
f) determining an algorithmic relationship between the buffer volume added and the analyte signal output; and
g) adjusting either the calibration curve or the output from a calibration curve to determining the analyte plasma concentration by accounting for the buffer volume added within the sample in accordance with steps e and f.

20. The method of claim 19, wherein the method further comprises determining the linear regression of the percent error and buffer volume using a five parameter logistic regression.

21. The method of claim 19, wherein the analyte is an anti-TNFα drug or an inflammatory protein.

22. The method of claim 19, wherein the anti-TNFα drug is a member selected from the group consisting of REMICADE™ (infliximab), INFLECTRA (Infliximab-dyyb), RENFLEXIS (Infliximab-abda), FLIXABI (Infliximab Biosimilar), REMSIMA (Infliximab Biosimilar), ENBREL™ (etanercept), HUMIRA™ (adalimumab), AMJEVITA (Adalimumab-atto), IMRALDI (Adalimumab Biosimilar), CYLTEZO (Adalimumab Biosimilar), HYRIMOZ (Adalimumab Biosimilar), HULIO (Adalimumab Biosimilar), CIMZIA® (certolizumab pegol), and combinations thereof.

23-43. (canceled)
